(19) United States
(12) Patent Application Publication
(10) Pub. No.: US 2005/0101885 A1
(43) Pub. Date: May 12, 2005

(54) Apparatus for determining the length of the endocervical canal

(76) Inventor: Gerald Mulvaney, Cary, NC (US)

Correspondence Address: Peter McLarty, 4137 Towne Green Circle, Addison, TX, 75001, US

(21) Appl. No.: 10/706,333

(22) Filed: Nov. 12, 2003

(51) Int. Cl. A61B 5/107

(52) U.S. Cl. 600/591

(57) ABSTRACT

A measuring portion (40) is attached to a flexible portion (10) allowing the measuring portion (40) to slide along the length of the flexible portion (10). The flexible portion comprises an expandable end (30) and a valvable end (50). The required lengths are determined from graduated markings of distance (20) along the length of the flexible portion (10).

### FIELD OF THE INVENTION

The invention is generally related to the field of gynecological instrumentation and more specifically to an apparatus for measuring the length of the uterine cavity and the endocervical canal.

### BACKGROUND OF THE INVENTION

The development of new gynecological procedures has resulted in the need for an accurate measure of the length of the cervix. Endometrial ablation is one such procedure that involves the destruction of a thin layer of the lining of the uterus. This thin layer is called the endometrium and is shed each month during a woman's menstrual period. This process is regulated by the hormones estrogen and progesterone. A normal cycle lasts twenty-eight days from the start of one period to the next. Normal flow lasts five to seven days and is equal to about a quarter cup of blood.

About one in five women have excessive menstrual bleeding. This is more common for women between the ages of forty-to-fifty who are approaching menopause but can present at any age. Excessive menstrual bleeding can lead to fatigue, anemia, and embarrassing accidents and can have a very negative effect on a woman's quality of life.

Treatment options for excessive menstrual bleeding include drug therapy which is the first line; these include oral contraceptives and also other hormones to help regulate the hormonal imbalances, D&C which involves scraping the lining of the uterus, hysterectomy and endometrial ablation.

Ablation will stop menses in most women. Other women will have light bleeding and very few may still have regular periods. The procedure is a very short and is done on an outpatient basis, with local anesthesia and light sedation. This makes for a quick recovery and minimal cramping post operatively. The techniques employed have evolved over time from the tedious rollerball and laser procedures to the more efficient and timely thermal, cryotherapy and electrical modalities.

Present techniques for performing ablation require an accurate measure of the length of the endocervical canal. This is a dimension that is not routinely measured by gynecologists and there is no instrument available that provides an accurate measurement. Presently a very crude and inaccurate method of measurement is utilized that, if performed incorrectly, can lead to perforation of the uterus, infection, excessive blood loss, and possible accumulation of blood in the uterus due to scarring. There is therefore a need for a safe and accurate apparatus and method for measuring the length of the endocervical canal. The instant invention addresses this need.

### SUMMARY OF THE INVENTION

The instant invention comprises a measuring apparatus for measuring the length of the uterine cavity and the endocervical canal. The measurement apparatus comprises a flexible tube with graduated markings for measuring distance. The markings can represent any established measure of distance such as centimeters, inches and fractions thereof. The tube comprises an expandable end with an adjustable cross-section. The other end of the tube comprises a valve and locking mechanism allowing the ingress and/or egress of liquids and/or gases into tube. A measuring portion is fitted over the tube that can be slid along the length of the tube allowing measurements to be taken. The measurements can be read directly from the graduated markings on the tube by determining the position of an edge of the measuring portion on the tube. In addition, the measuring portion can be used to mark the tube thereby allowing the measurement to be recorded.

In operation the expandable end of the measuring apparatus is inserted into the uterine cavity through the vagina and the cervix. At the point when the expandable end of the flexible tube is at the top of the uterine cavity (or fundus) the measuring portion is slid along the length of the flexible tube until it touches the cervix. The required length can then be measured by marking the flexible tube or by determining the position of the measuring portion along the flexible tube using the graduated markings along the tube.

To determine the length of the endocervical canal, the end of the flexible tube is expanded and the flexible tube slowly withdrawn from the uterine cavity until the expanded end rests against the end of the endocervical canal. At this point the measuring portion is slid along the length of the flexible tube until it rests against the cervix. The required length can then be measured by marking the flexible tube or by determining the position of the measuring portion along the flexible tube using the graduated markings along the tube. The measuring apparatus can be removed from the uterus and vagina by reducing the cross-section of the expanded end and slowly withdrawing the flexible tube from the uterus and vagina.

### BRIEF DESCRIPTION OF THE DRAWINGS

FIG. 1(a). A diagram of an embodiment of the measuring apparatus according to the instant invention.

FIG. 1(b). A cross-section of the measuring apparatus taken through the plane Z-Z′ shown in FIG. 1(a).

FIG. 2(a) A top view of an embodiment of the measuring and/or marking component of the apparatus of the instant invention.

FIG. 2(b). A cross-sectional view of the embodiment of the measuring and/or marking guide component of the apparatus shown in FIG. 2(a).

FIGS. 3(a)-FIG. 3(b). Diagrams of an embodiment of tip of the measurement apparatus in the (a) normal state and the (b) inflated or expanded state.

FIG. 4. A diagram showing an embodiment of a valve assembly in the base of the measuring apparatus.

FIG. 5(a)-FIG. 5(b). Diagrams illustrating the measuring apparatus according to an embodiment of the instant invention.

Common reference numerals are used throughout the figures to represent like or similar features. The figures are not drawn to scale and are merely provided for illustrative purposes.

### DETAILED DESCRIPTION OF THE INVENTION

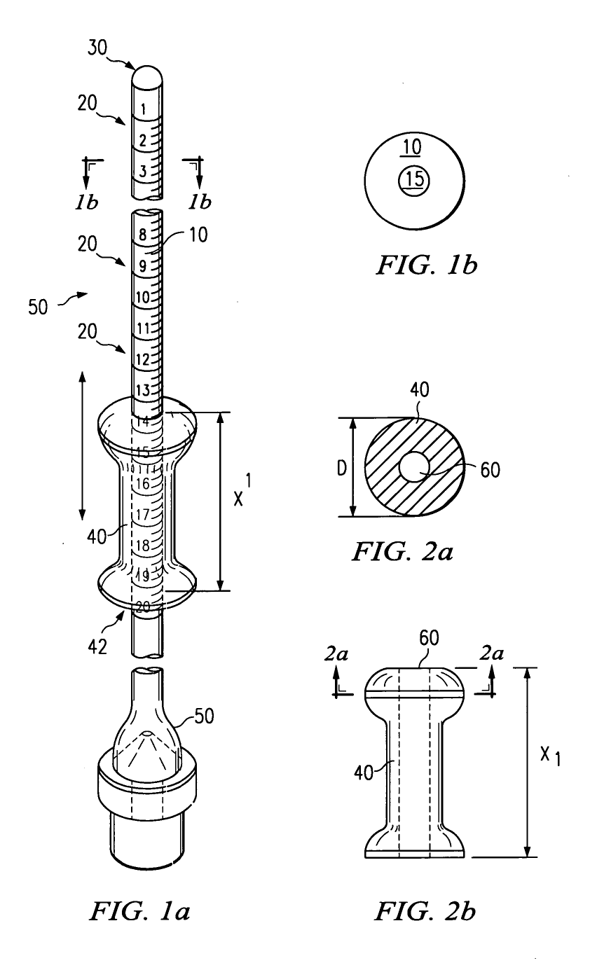

The invention will be described by referring to FIGS. 1 through FIG. 5(b). The Figures illustrate an embodiment of an apparatus and method for using the apparatus to measure the length of the uterine cavity and the length of the endocervical canal.

Shown in FIG. 1 is an embodiment of an apparatus 5 to measure the length of the uterine cavity and the length of the endocervical canal. The measurement apparatus comprises a flexible portion 10, an expandable end portion 30, a valvable end portion 50, and a measuring/marking portion 40. In an embodiment of the instant invention the cross-section of the flexible portion 10 is approximately 3 mm across but can be anywhere from 0.2 mm to 8 mm without departing from the scope of the instant invention. The flexible portion can be formed using any suitable flexible material such as various types of rubber or plastic. In addition, other combinations of flexible materials can be used to form the flexible portion 10. The requirement for the flexibility or rigidity of the flexible portion 10 is such that it is flexible enough to facilitate insertion from outside the vaginal opening through the cervix and into the uterine cavity. The flexible portion 10 should however be rigid enough to maintain its shape in the uterine cavity thereby allowing accurate measurements to be obtained. Markings representing distance 20 are placed along the length of the flexible portion 10 to facilitate the taking of the measurements. In various embodiments the markings 20 can represent inches (as well as any usable fraction of an inch) or centimeters (as well as any usable fraction of a centimeter such as millimeters etc.) or any other suitable measure of distance. The markings start at a zero measure at the expandable end 3 and increase towards the valvable end 50 of the flexible portion 10. In cross-section the flexible portion 10 can be circular, square, rectangular, octagonal, triangular, or any other suitable shape. Shown in FIG. 1(b) is an embodiment of a cross-section of the flexible portion 10 taken along the line ZZ′ as shown in FIG. 1(a) for an embodiment of the instant invention. As shown in FIG. 1(b) the flexible portion has a hollow center 15 allowing gas or liquid to be used to inflate the end 30 of the flexible portion. The hollow center runs the entire length of the flexible portion 10 from the valvable end 50 to the expandable end 30.

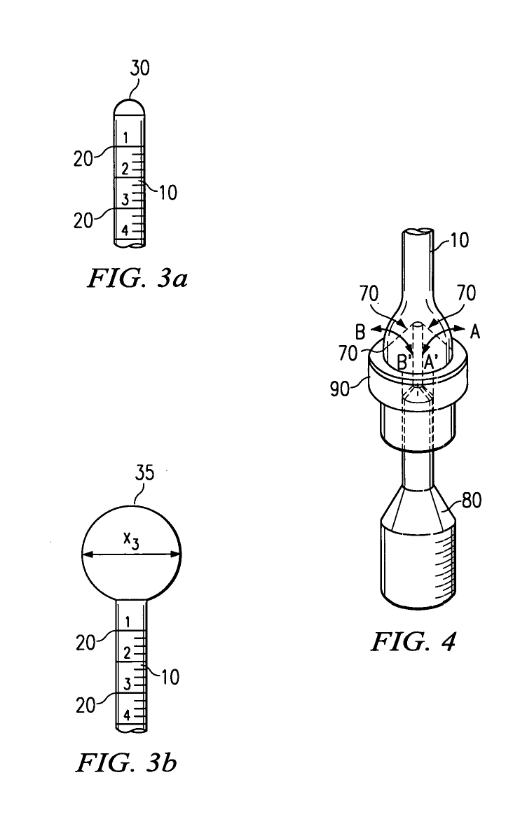

The measuring apparatus shown in FIG. 1(a) comprises an expandable end portion 30. An embodiment of the expandable end portion is shown in FIG. 3(a) and FIG. 3(b). As shown in FIG. 3(a) in the unexpanded state the expandable end 30 of the flexible portion 10 is approximately the same size or dimension of the flexible portion 10. On the introduction of a gas or liquid into the flexible portion 10 through the valvable end 50 as will be described below, the expandable end 30 will expand as shown in FIG. 3(b). The expanded end 35 will now have a cross-section x₃ that is greater than the unexpanded cross-section of the expandable end 30. The embodiment of the expandable end 30 shown in FIGS. 3(a) and 3(b) represent one of many embodiments. In other embodiments a collapsible portion is expanded to increase the cross-section of the expandable end 30.

The measuring apparatus shown in FIG. 1(a) further comprises a measuring or marking portion (herein after referred to as the measuring portion) 40 that moves along the flexible portion 10. An embodiment of the measuring portion is shown in FIG. 2(a) and FIG. 2(b). A top view of an embodiment of the measuring portion is shown in FIG. 2(a). In the embodiment shown in FIG. 2(a) the measuring portion 40 is circular with a hollow center portion 60 that allows the measuring portion 40 to move along the length of the flexible portion 10 as shown in FIG. 1(a). As shown in FIG. 2(b), the measuring portion 40 can be a length x₁ that allows the position of the measuring portion along the flexible portion 10 to be easily read. An example of this is shown in FIG. 1(a) where the distance of the end of the measuring portion 42 from the expandable end 30 can be read from the markings 20 on the flexible portion 10. It should be noted that the measuring portion can be any shape that allows the distance of the measuring portion 40 from the expandable end 30 to be easily obtained. In a further embodiment the measuring portion can be used to mark the flexible portion thereby recording the distance from the expandable end 30 of the flexible portion to the position of the measuring portion at the time the mark was made.

The measuring apparatus shown in FIG. 1(a) further comprises a valvable end 50. The valvable end 50 is positioned along the flexible portion 10 at the end opposite to that of the expandable end 30. The valvable end 50 allows gases and/or liquids to enter the flexible portion 10 to expand the expandable end 30. Once expanded the valvable end 50 allows gas and/or liquids to leave the flexible portion 10 thereby allowing the expandable end 30 to return to its unexpanded size. An embodiment of the valvable end 50 is shown in FIG. 4. As shown in FIG. 4, the valvable end 50 comprises a locking mechanism 90 that locks an external device 80 in place. In an embodiment of the instant invention the external device 80 is a syringe that is used to inject liquid and/or gas into the flexible portion 10 to expand the expandable end 30. In addition the syringe 80 can allow liquid and/or gas to leave the flexible portion 10 allowing the expanded end 30 to contract. Optional valves 70 that move along the arc AA′ and the arc BB′ can be used to hold the liquid and/or gas in the flexible portion 10 if the external device 80 is removed.

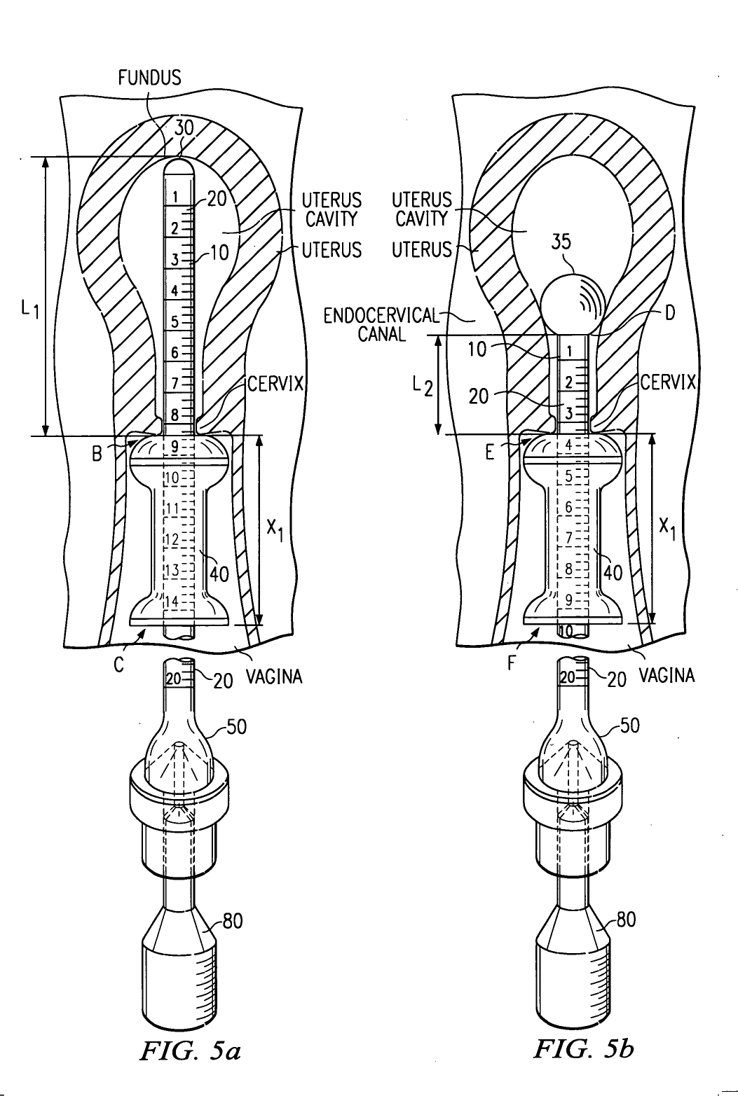

Illustrated in FIG. 5(a) and FIG. 5(b) is an embodiment of a method for using the measuring apparatus to measure the length of the uterus and the endocervical canal. As shown in FIG. 5(a) the flexible portion 10 is inserted through the vagina and the cervix and into the uterine cavity. To measure the length of the uterine cavity the flexible portion 10 is inserted until the expandable end 30 of the flexible portion 10 touches the fundus or the end of the uterine cavity. The expandable end of the flexible portion is the zero point of the measurement. The measuring portion 40 is then slid along the length of the flexible portion 10 until it rests against the cervix as shown at point B in FIG. 5(a). At this point the measuring portion 40 can be used to mark the flexible portion 10 or the measurement can be read from the end of the measurement portion 40 at point C as shown in FIG. 5(a). For the case where the measurement is read at point C, the length of the uterus L₁ is found by subtracting the length of the measuring portion x₁ from the length read at point C.

To measure the length of the endocervical canal, the expandable end 30 of the flexible portion 10 is expanded and the flexible portion 10 is slowly withdrawn from the uterine cavity. The flexible portion 10 is slowly withdrawn the expanded end 35 rests against the end of the endocervical canal as shown at point D in FIG. 5(b). It is important that the size of the expanded end 35 be such that it does not easily pass through the endocervical canal. There should be enough resistance to the expanded end 35 passing through the endocervical canal to signal the user that the expanded end 35 is at the edge D of the endocervical canal. When the expanded end 35 is at the edge D of the endocervical canal, the expanded end 35 becomes the zero point of the measurement, and the measuring portion 40 is slid along the flexible portion 10 to the edge of the cervix (point E) as shown in FIG. 5(b). To extract the length of the endocervical canal the measuring portion 40 can be used to mark the flexible portion 10. Alternatively, the measurement can be read from the end of the measuring portion 40 at point F as shown in FIG. 5(b). In the case where the measurement is read at point F, the length of the endocervical canal L₂ is found by subtracting the length X₁ of the measuring portion 40 from the length measured at point F in FIG. 5(b). Following the measurement of the length of the endocervical canal, the size of the expanded end 35 is reduced and the flexible portion 10 is slowly removed from the uterine cavity through the cervix and the vagina.

Although the present invention has been described in detail, various changes and modifications may be suggested to one skilled in the art. It is intended that the present invention encompass such changes and modifications as falling within the scope of the appended claims.

1. A gynecological measuring apparatus, comprising:
a flexible portion comprising an expandable end;
a measuring portion on said flexible portion wherein said measuring portion can slide along said flexible portion; and
a valvable end on said flexible portion.

2. The measuring apparatus of claim 1 wherein said flexible portion has markings representing distance.

3. The measuring apparatus of claim 1 wherein said flexible portion is a tube.

3. The measuring apparatus of claim 1 wherein said expandable end can increase in size.

4. The measuring apparatus of claim 1 wherein said valvable end comprises a locking mechanism for locking an external device in place.

5. The measuring apparatus of claim 3 wherein said valvable end allows liquids to enter said flexible portion and increase the size of said expandable end.

6. The measuring apparatus of claim 3 wherein said valvable end allows gases to enter said flexible portion and increase the size of said expandable end.

7. The measuring apparatus of claim 1 wherein said measuring portion is used to mark said flexible portion.

8. A method for measuring a length of a uterus, comprising:
inserting a flexible tube into a uterine cavity until an end of said flexible tube is at a fundus or top of the uterine cavity;
sliding a measuring portion along said flexible tube until said measuring portion is against a cervix; and
determining the length of the uterus by determining a distance along the flexible tube from said measuring portion to the end of said flexible tube.

9. The method of claim 8 wherein the determining of the length of the uterus comprises obtaining a position of the measuring portion from graduated markings of distance on said flexible tube.

10. The method of claim 8 wherein the determining of the length of the uterus comprises obtaining a position of the measuring portion from a mark made by said measuring portion on said flexible tube.

11. A method for measuring a length of an endocervical canal, comprising:
inserting a first end of a flexible tube through said endocervical canal and into a uterine cavity;
expanding said first end of said flexible tube;
withdrawing said flexible tube from said uterine cavity until said expanded first end of said flexible tube is against an end of said endocervical canal;
sliding a measuring portion along said flexible tube until said measuring portion is against a cervix; and
determining the length of the endocervical canal by determining a distance along the flexible tube from the measuring portion to the expanded end of said flexible tube.

12. The method of claim 11 wherein the determining of the length of the endocervical canal comprises obtaining a position of the measuring portion from graduated markings of distance on said flexible tube.

13. The method of claim 11 wherein the determining of the length of the endocervical canal comprises obtaining a position of the measuring portion from a mark made by said measuring portion on said flexible tube.
